(19) United States
(12) Patent Application Publication
(10) Pub. No.: US 2008/0154304 A1
(43) Pub. Date: Jun. 26, 2008

(54) SYSTEM AND METHOD FOR ACCESSING A TISSUE STRUCTURE

(75) Inventors: Darren Crawford, Los Altos, CA (US); George Delli-Santi, Scotts Valley, CA (US)

Correspondence Address: ARTHROCARE CORPORATION, 7500 Rialto Boulevard, Building Two, Suite 100, Austin, TX, 78735-8532, US

(73) Assignee: ArthroCare Corporation, Austin, TX (US)

(21) Appl. No.: 11/614,777

(22) Filed: Dec. 21, 2006

(51) Int. Cl. A61B 17/70; A61B 17/17

(52) U.S. Cl. 606/246; 206/572; 606/96; 606/93

(57) ABSTRACT

A tool system for facilitating medical procedure including accessing a selected bone structure or tissue structure includes a cannula and an obturator with a drill section formed on its distal end. Additionally a drill guide may protrude from the distal end of the obturator. The disclosed tool system may be used to penetrate the cutaneous layers of a patient until the drill guide penetrates the exterior surface of a target bone structure such as a vertebral body. The system may then be rotated and torqued to allow the drill section of the obturator to bore into the bone structure. As the system advances, the cannula translates forward within the bore created by the drill section until a desired placement of the cannula is achieved.

### FIELD OF THE INVENTION

The present invention relates generally to an apparatus for accessing bone or tissue structures and, more particularly, to an obturator for use with a cannula to penetrate bone or other tissue for therapeutic applications.

### BACKGROUND OF THE INVENTION

A number of techniques and apparatus have been developed for accessing target areas of bone or tissue within a patient. For example, procedures such as vertebroplasty and kyphoplasty require the insertion of an access device such as a cannula into a target area of bone to achieve access to an implantation site. In vertebroplasty, cancellous bone of the vertebrae may then be supplemented with “bone cement,” e.g., polymethylmethacrylate (PMMA) or another material, in order to provide for anterior and posterior stabilization of the spine. In kyphoplasty, an expandable device such as a balloon is inserted into the interior of the vertebra and expanded. Following removal of the expandable device, the resulting void is typically filled with bone cement to promote stabilization of the vertebrae. Vertebroplasty and kyphoplasty are desirable from the standpoint that they are minimally invasive as compared to conventional procedures requiring surgically exposing a tissue site that is to be supplemented with bone cement.

Several procedures are known for accessing a desired site in the cancellous bone of a vertebral body, or substantially any other cancellous bone, to deliver an expandable device and bone cement or another suitable hard tissue implant material to stabilize, or build up, a target site as taught by U.S. Pat. No. 6,280,456, U.S. Pat. No. 6,248,110, U.S. Pat. No. 5,108,404, and U.S. Pat. No. 4,969,888, which are each incorporated herein by reference.

In another known example, a standard 11 gauge Jamshidi needle including a cannula and an internal stylet may be used to pierce the cutaneous layers of a patient as well as the hard cortical bone of a vertebral body to access the cancellous bone within the interior of the vertebral body. In order to provide sufficient force, a mallet is typically used to urge the cannula and stylet through the cortical bone.

To gain access to a hard tissue implantation site, as described in U.S. Pat. Nos. 6,019,776 and 6,933,411, which are each incorporated herein by reference, a straight needle or cannula in combination with a stylet may be employed. As discussed therein, a stylet incorporating self-tapping threads may be utilized to penetrate the cortical bone of the vertebra. Once access is achieved and the stylet is removed from the cannula, bone cement may be delivered through the cannula for the purposes of filling the hard tissue implantation site.

In another example, a small diameter cannula (such as an 11 gauge cannula) may be first used to access a target tissue such as a vertebral body. Following initial placement, the stylet of the small diameter cannula is removed and replaced with a guide wire. The small diameter cannula may then be removed, leaving the guide wire in place. A cannulated drill may then be slid over the guide wire and advanced to the target site and rotated to expand the existing access area. An access cannula (such as an 8 gauge cannula) may then be inserted over the cannulated drill. Following placement of the access cannula, the cannulated drill and guide wire may be removed, thereby providing access to the target site via the access cannula.

Presently available methods and instrumentation for accessing target areas have a number of drawbacks. Cannula assemblies driven into a vertebral body with a mallet do not provide for controlled advancement of the cannula. Use of multiple cannulae, a guide wire and a cannulated drill requires a significant number of steps and instruments, as well as time, in order to gain access to the target area. Cannula assemblies using stylets with self-tapping threads may only advance the cannula through the outer cortical shell of a bone, after which the threaded stylet loses its purchase in bone and no longer functions to advance the cannula into the cancellous interior of the bone.

### SUMMARY OF THE INVENTION

Therefore a need has arisen for an improved system and method for accessing a bone structure.

A further need has arisen for an improved system and method for accessing a vertebral body in a single step.

The present invention relates to performing a medical procedure including accessing a bone structure that reduces or eliminates the problems and drawbacks of existing systems and methods. A tool system including a cannula and an obturator with a drill section formed on its distal end is disclosed. Additionally a drill guide may protrude from the distal end of the obturator. The disclosed tool system may be used to penetrate the cutaneous layers of a patient until the drill guide penetrates the exterior surface of a target bone structure such as a vertebral body. The system may then be rotated and torqued to allow the drill section of the obturator to bore into the bone structure. As the system advances, the cannula translates forward within the bore created by the drill section until a desired placement of the cannula is achieved.

In one aspect an obturator is disclosed including an elongated body with a longitudinal axis, a diameter, a proximal end and a distal end. The obturator also includes a drill section formed on the distal end of the elongated body and a drill guide extending from the distal end of the elongated body and adapted to penetrate a bone structure.

In another aspect a tool system is disclosed that includes a cannula and an obturator. The obturator has an elongated body with a proximal end and a distal end and is adapted to fit within and be removed from the cannula. A drill section is formed on a portion of the distal end of the elongated body and a drill guide extends from the distal end of the elongated body.

In yet another aspect, a medical procedure to be performed on a body is disclosed. The medical procedure includes slidably engaging an obturator within a cannula, wherein the obturator includes an elongated body with a drill section formed on the distal end of the elongated body and a drill guide extending from the distal end of the elongated body. The procedure also includes penetrating tissue with the assembled cannula system and advancing the assembled cannula system until the drill guide penetrates a bone structure. The assembled cannula is then advanced to a target location within the bone structure by rotating the cannula system, thereby causing the drill portion to drill into the bone structure.

In yet another aspect a kit is disclosed including a cannula, an obturator and a bone cement injection system. The obturator is adapted to fit within and be removed from the cannula. The obturator includes an elongated body with a proximal end and a distal end, a drill section formed on the distal end, and a drill guide extending from the distal end of the elongated body. The bone cement injection system includes a connector adapted to fluidly connect the delivery system with the cannula such that bone cement may be delivered from the delivery system through the cannula to a target site.

The present disclosure includes a number of important technical advantages. One technical advantage is the provision of an obturator having a drill section. The obturator of the present invention facilitates controlled advancement and positioning of an access cannula in a target area in a single step. Additional advantages will be apparent to those of skill in the art and from the figures, description and claims provided herein.

### BRIEF DESCRIPTION OF THE DRAWINGS

The invention may best be understood by reference to the following description taken in conjunction with the accompanying drawings in which:

FIG. 1 shows a tool system including a cannula and obturator according to teachings of the present disclosure;

FIG. 2 shows a tool system including a cannula having a threaded portion and an obturator according to teachings of the present disclosure;

FIGS. 3A and 3B show a cross sectional view of an obturator according to teachings of the present disclosure;

FIGS. 4A through 4C show side views of obturator drill section configurations according to teachings of the present disclosure;

FIGS. 5A through 5D show a tool system accessing a vertebral body according to teachings of the present disclosure;

FIG. 6 shows a tool system for treatment of a vertebral body including a tissue treatment device according to teachings of the present disclosure;

FIGS. 7A and 7B show a tool system including an expandable device according to teachings of the present disclosure;

FIG. 8 shows a tool system for treating a vertebral body including a cement delivery system according to teachings of the present disclosure;

FIG. 9 shows an access kit according to teachings of the present disclosure; and

FIG. 10 shows a flow diagram of a method according to teachings of the present disclosure.

### DETAILED DESCRIPTION

While the present invention is described in detail, it is to be understood that this invention is not limited to particular variations set forth herein as various changes or modifications may be made to the embodiments described and equivalents may be substituted without departing from the spirit and scope of the invention. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, many modifications may be made to adapt a particular situation, material, composition of matter, process, process act(s) or step(s) to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Last, it is to be appreciated that unless defined otherwise, all technical and scientific terms used herein have the same meaning as commonly understood by one of ordinary skill in the art to which this invention belongs.

The systems of the present invention may be configured for use in any suitable minimally invasive procedure that involves accessing a target site within a patient. As discussed herein, the subject systems are particularly suitable for accessing and treating vertebral bodies. In certain embodiments, the system further includes a treatment device suitably configured for treating a degenerative intervertebral disc.

The treatment device of the present invention may have a variety of configurations and characteristics as described below. However, one variation of the invention employs a tissue treatment device using Coblation® technology. The assignee of the present invention developed Coblation® technology. Coblation® technology involves the application of a high frequency voltage difference between one or more active electrode(s) and one or more return electrode(s) to develop high electric field intensities in the vicinity of the target tissue. The high electric field intensities may be generated by applying a high frequency voltage that is sufficient to vaporize an electrically conductive fluid over at least a portion of the active electrode(s) in the region between the tip of the active electrode(s) and the target tissue. The electrically conductive fluid may be a liquid or gas, such as isotonic saline, blood, extracelluar or intracellular fluid, delivered to, or already present at, the target site, or a viscous fluid, such as a gel, applied to the target site.

When the conductive fluid is heated enough such that atoms vaporize off the surface faster than they recondense, a gas is formed. When the gas is sufficiently heated such that the atoms collide with each other causing a release of electrons in the process, an ionized gas or plasma is formed (the so-called “fourth state of matter”). Generally speaking, plasmas may be formed by heating a gas and ionizing the gas by driving an electric current through it, or by shining radio waves into the gas. These methods of plasma formation give energy to free electrons in the plasma directly, and then electron-atom collisions liberate more electrons, and the process cascades until the desired degree of ionization is achieved. A more complete description of plasma can be found in Plasma Physics, by R. J. Goldston and P. H. Rutherford of the Plasma Physics Laboratory of Princeton University (1995), the complete disclosure of which is incorporated herein by reference.

As the density of the plasma or vapor layer becomes sufficiently low (i.e., less than approximately 1020 atoms/cm3 for aqueous solutions), the electron mean free path increases to enable subsequently injected electrons to cause impact ionization within the vapor layer. Once the ionic particles in the plasma layer have sufficient energy, they accelerate towards the target tissue. Energy evolved by the energetic electrons (e.g., 3.5 eV to 5 eV) can subsequently bombard a molecule and break its bonds, dissociating a molecule into free radicals, which then combine into final gaseous or liquid species. Often, the electrons carry the electrical current or absorb the radio waves and, therefore, are hotter than the ions. Thus, the electrons, which are carried away from the tissue towards the return electrode, carry most of the plasma's heat with them, allowing the ions to break apart the tissue molecules in a substantially non-thermal manner.

By means of this molecular dissociation (rather than thermal evaporation or carbonization), the target tissue structure is volumetrically removed through molecular disintegration of larger organic molecules into smaller molecules and/or atoms, such as hydrogen, oxygen, oxides of carbon, hydrocarbons and nitrogen compounds. This molecular disintegration completely removes the tissue structure, as opposed to dehydrating the tissue material by the removal of liquid within the cells of the tissue and extracellular fluids, as is typically the case with electrosurgical desiccation and vaporization. A more detailed description of this phenomena can be found in commonly assigned U.S. Pat. No. 5,697,882 the complete disclosure of which is incorporated herein by reference.

The amount of energy produced by the Coblation® device may be varied by adjusting a variety of factors, such as: the number of active electrodes; electrode size and spacing; electrode surface area; asperities and sharp edges on the electrode surfaces; electrode materials; applied voltage and power; current limiting means, such as inductors; electrical conductivity of the fluid in contact with the electrodes; density of the fluid; and other factors. Accordingly, these factors can be manipulated to control the energy level of the excited electrons.

The active electrode(s) of a Coblation® device may be supported within or by an inorganic insulating support positioned near the distal end of the instrument shaft. The return electrode may be located on the instrument shaft, on another instrument or on the external surface of the patient (i.e., a dispersive pad). The proximal end of the instrument(s) will include the appropriate electrical connections for coupling the return electrode(s) and the active electrode(s) to a high frequency power supply, such as an electrosurgical generator.

A more detailed discussion of spinal applications and devices using Coblation® technology may be found as follows: issued U.S. Pat. Nos. 6,105,581; 6,283,961; 6,264,651; 6,277,112; 6,322,549; 6,045,532; 6,264,650; 6,464,695; 6,468,274; 6,468,270; 6,500,173; 6,602,248; 6,772,012; and 7,070,596 each of which is incorporated by reference, and pending U.S. patent applications Ser. No. 09/747,311 filed Dec. 20, 2000 and Ser. No. 10/656,597 filed Sep. 5, 2003 both of which are incorporated by reference.

The following discussion is an example of the inventive method as applied to a vertebroplasty or kyphoplasty procedure. It is understood that the invention is not limited to vertebroplasty and kyphoplasty procedures. Instead, the cannula and obturator configurations disclosed herein may be used in any procedure where it is desired to establish cannular access to a bone structure or another tissue structure. Moreover, other treatment modalities (e.g., chemical, other electrosurgical devices, etc.) may be used in the inventive method either in place of the Coblation® technology or in addition thereto.

In further describing the subject invention, the subject devices and systems will be described first followed by a description of kits which include the subject devices, followed by a description of subject methods of the present disclosure.

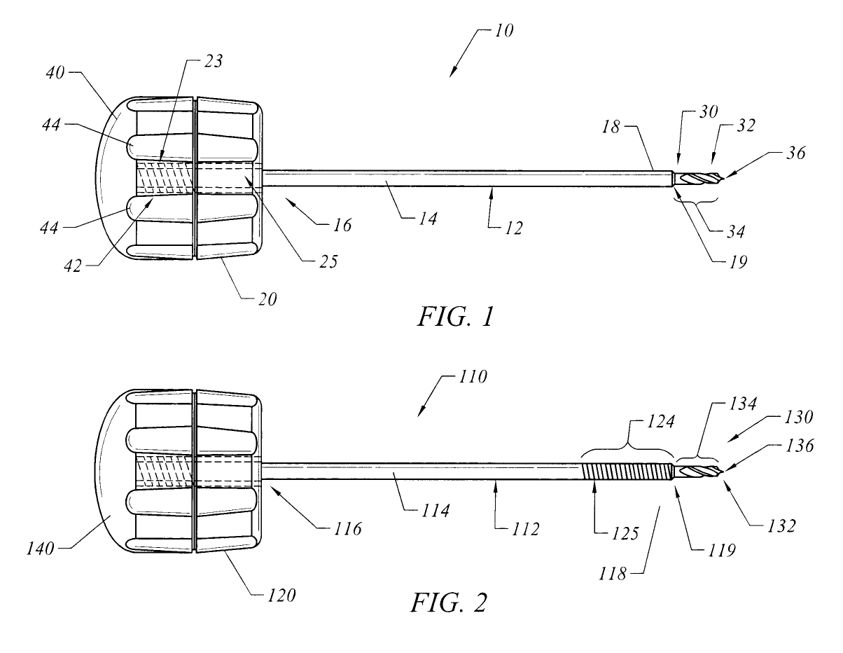

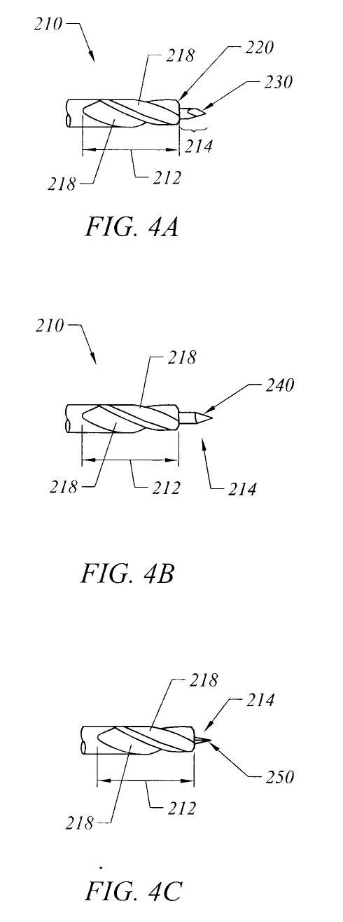

Now referring to FIG. 1, an assembled cannula system, which may also be referred to as a “tool system” herein, is generally indicated at reference numeral 10. Cannula system 10 generally includes cannula 12 and obturator 30. As discussed herein, obturator 30 may also be referred to as a stylet or a trocar. Cannula 12 generally includes a tubular cannula body 14 having a proximal end 16 and a distal end 18. A cannula handle 20 is attached at the proximal end 16 of cannula body 14. Obturator 30 includes a distal end 32 with a drill section 34 formed thereon. A drill guide 36 preferably protrudes from distal end 32 of obturator 30. Obturator 30 is generally sized to fit within and to be removed from the lumen defined within the interior of cannula 12 and to effectively block the opening of said lumen. Obturator 30 also includes a handle 40 formed on the proximal end thereof (as shown in FIGS. 3A and 3B, below).

Obturator handle 40 is removably coupled with cannula handle 20. In the present embodiment cannula handle 20 includes an interface structure including an externally threaded column 23 extending from the cannula handle 20 and adapted to couple with a threaded interface structure 42 formed within obturator handle 40. Interface structure includes a longitudinal bore 25 substantially coaxial with the lumen of cannula 12. In alternate embodiments cannula handle 20 and obturator handle 40 may incorporate any suitable structure for removably coupling handles 20 and 40. In the present embodiment cannula handle 20 and obturator handle 40 each have a generally cylindrical outer surface with exterior grooves 44 formed thereon. In alternative embodiments, cannula handle 20 and obturator handle 40 may form a T-shaped handle or any other suitable handle configuration well known in the medical arts.

In one example embodiment, obturator 30 may be formed from pre-hardened 17-4 stainless steel where drill section 34 is machined and electropolished. Also, cannula 12 may be formed from 304 Stainless Steel and handles 20 and 40 may be formed from a plastic material, such as, trogamid. In alternate embodiments obturator 30, cannula 12 and handle portions 20 and 40 may be formed using any suitable material. For example obturator 30 and cannula 12 may be made from 316 stainless steel, precipitation-hardened stainless steel alloys (such as 17-7 or 17-4), titanium alloys, or cobalt-chromium alloys; the handle portions 20 and 40 from polyacetal, ABS, polycarbonate, polyphenylene sulfide, PEEK, Cyclic Olefin Copolymers (COC, e.g. Topas®), and other structural engineering plastics, including fiber-reinforced resins and self-reinforcing polymers (e.g. Tecamax™ SRP, PrimoSpire™ SRP).

In the above example embodiment cannula 12 may have an outer diameter of about 0.176 inches (not expressly shown), an inner diameter of about 0.145 inches (not expressly shown) and obturator 30 may have an outer diameter 60 of about 0.140 inches.

When assembled, distal end 32 of obturator 30 extends beyond the distal end 18 of cannula 12, thereby exposing at least a portion of drill section 34. In one embodiment obturator 30 extends beyond distal end 18 of cannula 12 about 0.5 inches. In alternate embodiments obturator 30 may extend beyond distal end 18 in the range between about 0.25 inches and 0.75 inches. As discussed below, drill section 34 includes one or more flutes formed thereon adapted for drilling through tissue including bone tissue. Drill guide 36 preferably includes a distal pointed end adapted to establish initial penetration into a target bone structure. Drill guide 36 is preferably disposed adjacent to drill section 34. Following initial penetration of drill guide 36 within a bone structure, system 10 may be rotated and advanced by a user, causing drill section 34 to form a bore with the bone structure. As system 10 translates into the bone structure, distal end 18 of cannula 14 enters the newly formed bore. It is noted that cannula 12 has a slightly larger diameter than obturator 30; as obturator 30 advances it may preferably cause the bore created by drill section 34 to expand to accommodate the diameter of cannula 12. In the present embodiment end surface 19 of obturator 12 comprises a sloped transition surface between the inner diameter and the outer diameter to facilitate entry of cannula 12 into the bore formed by drill section 34.

System 10 may preferably be viewed using fluoroscopy or another suitable visualization technology during placement. A user may preferably continue to rotate and advance system 10 until a desired placement is determined via a visualization technology. Following placement, obturator 30 is preferably removed from system 10 while leaving cannula 12 in place to provide access to the target location. In one particular embodiment, system 10 may be used to establish transpedicular access within a vertebral body as described below.

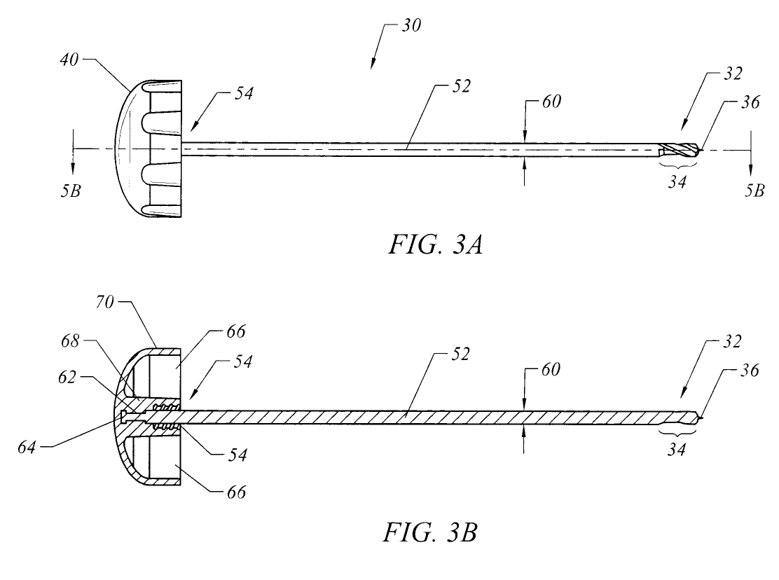

Now referring to FIG. 2, a cannula system 110 is disclosed generally corresponding to system 10 of FIG. 1 and also incorporating a threaded cannula 112. Cannula 112 comprises a tubular elongate body 114 having a proximal end 116 and a distal end 118. A handle 120 is attached at the proximal end 116 of cannula body 114. Additionally, cannula 112 comprises threaded portion 124 formed on the distal end 118 thereof. Threaded portion 124 includes threads 125 formed on the exterior surface of cannula body 114. Threaded portion 124 may comprise any suitable geometry or configuration suitable to facilitate the penetration of threaded portion 124 into a bone structure such as a vertebral body. In a particular embodiment threaded portion 24 may correspond with the threaded cannulas taught by U.S. patent application Ser. No. 11/022,062 which is incorporated herein by reference.

System 110 further includes an obturator 130 adapted to be inserted within the lumen of cannula 112 and to effectively block the opening of cannula 112 during placement. Obturator 130 also comprises a drill section 134 formed at the distal end 132 of obturator 130 as well as a drill guide 136 protruding therefrom.

As system 110 advances into a bone structure such as a vertebral body, threaded portion 124 follows the path directly behind drill portion 134. As system 110 is rotated, drill section 134 forms a bore in the target bone structure. Subsequently, threaded portion 124 may preferably engage the newly formed bore, facilitating both the controlled advancement of system 110 and a stable interface between cannula 112 and the target bone structure.

In a preferred embodiment, the pitch of the flutes of drill section 134 have a steeper pitch as compared with the threads 125 of threaded portion of cannula 112. As device 110 advances into a bone structure and threads 125 engage bone, threads 125 act to control the rate of advancement. In this manner the difference in pitch between cannula threads 125 and the flutes of drill section 134 ensures that the flutes of drill section 134 act in a cutting or drilling manner to remove tissue and prevents the flutes from acts similar to threads.

Now referring to FIG. 3A, a side view of an obturator assembly, generally depicted at 30 is shown. Obturator 30 includes an elongate obturator body 52 formed along longitudinal axis 58 having a proximal end 54, a distal end 32 and a diameter 60. Diameter 60 is selected to allow obturator 30 to slide in and out of a corresponding cannula and to effectively block the opening of the cannula (such as cannulas 12 and 112 as shown in FIGS. 1 and 2). Obturator 30 further includes drill section 34 formed on the distal end 32 thereof. Additionally, drill guide 36 protrudes from the distal end 32 of obturator 30. In the present embodiment drill section 34 and drill guide 36 are generally coaxially aligned with longitudinal axis 58.

As shown in FIG. 3B, a cut away view of obturator assembly 30 is shown, wherein handle 40 is attached to proximal end 54 of elongated body 52. In the present embodiment handle 40 comprises an outer body 70 and a central structure 68, leaving an open section or void 66 therebetween. Central structure 68 generally extends from a central portion of outer body 70 along longitudinal axis 58. Central structure 68 is adapted to interface with distal end 54 of obturator 50. In the present embodiment distal end 54 of obturator 50 includes a narrow portion 62 and an end structure 64 formed thereon. In this embodiment narrow portion 62 includes a section having a smaller diameter than diameter 60 and end structure 64 is formed proximate to narrow portion 62 and has a diameter substantially equal to diameter 60. The geometry and surfaces of narrow portion 62 and end structure 64 may preferably promote the secure attachment of distal end 54 of elongate body 52 to handle 40. Additionally, central structure 68 includes interface structure 42 which facilitates selective coupling with a threaded portion of a corresponding cannula handle as discussed above.

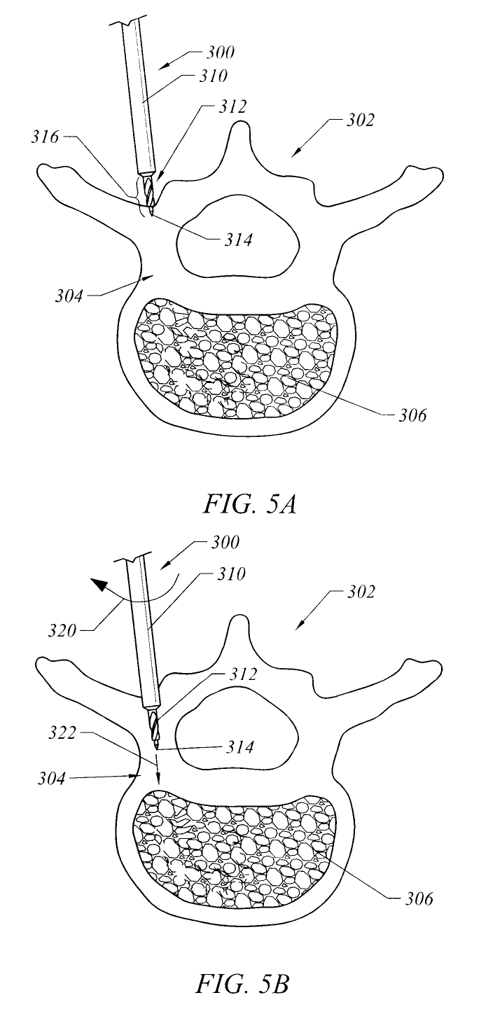

Now referring to FIGS. 4A-4C showing exemplary embodiments of portions of distal end 210 of an obturator (such as obturators 30 or 130 in FIGS. 1 and 2). Distal end 210 includes a drill section 212 having flutes 218 formed therein. In one embodiment drill section 212 has a length between about 0.25 inches and 0.75 inches. In a particular embodiment drill section 212 may have a length of approximately 0.5 inches; in another embodiment drill section 212 may have a length of approximately 0.45 inches. Additionally, in one particular embodiment flutes 218 have a helix angle of approximately 20 degrees. In alternate embodiments flutes 218 have a helix angle between, for example, about 10 degrees and about 30 degrees. Additionally, flutes 218 may have a flute depth, for example, of between about 0.02 inches and about 0.04 inches. In one embodiment flutes 218 have a flute depth of about 0.03 inches. Obturator distal end 210 further comprises a drill point 220 section transitioning between the diameter of distal end of drill section 212 and the smaller diameter of drill guide 230. In one embodiment, drill point 220 comprises a point angle of about 120°. In alternate embodiments, drill point 220 may comprise any suitable drill point angle including a drill point angle between about 100° and 140°. In other alternate embodiments obturator distal end 210 may not incorporate a drill point.

In the embodiment of FIG. 4A, drill guide 214 protrudes from the distal end of drill section 212 and incorporates a diamond-shaped tip 230. Drill guide 214 has a length of between about 0.05 inches and about 0.25 inches. In a particular embodiment, drill guide has a length of about 11 inches. In alternate embodiments drill guide 214 may comprise any suitable length for establishing initial penetration into a target bone structure. As shown in the embodiment of FIG. 4B, drill guide 214 includes a conical tip 240 protruding from drill section 212. As shown in the embodiment of FIG. 4C, drill guide 214 comprises a narrow, needle-shaped tip 250 protruding from drill section 212. This needle-shaped tip 250, as well any of drill guides discussed herein, may be of a pyramid, diamond, trocar, conical, or other similar design that results in a sharp, penetrating geometry.

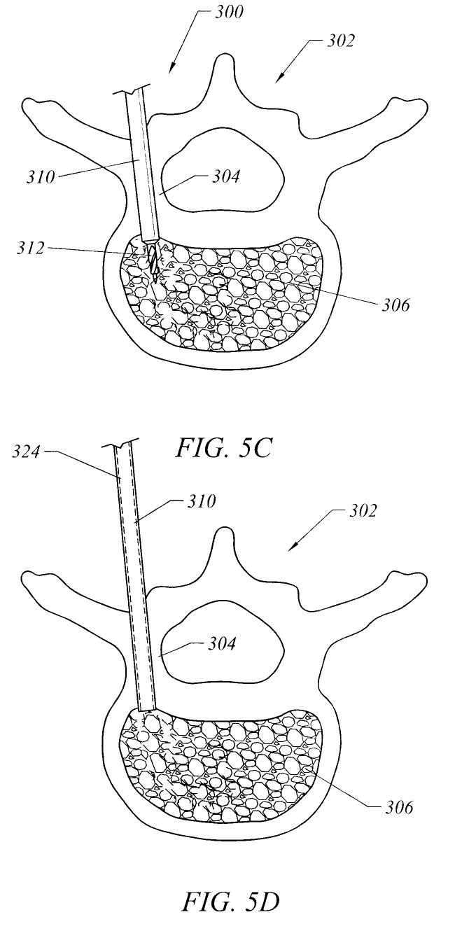

Now referring to FIG. 5A, a depiction of a tool system 300 (with portions removed) is shown entering a bone structure 302 to perform a medical procedure in accordance with teachings of the present disclosure. In the present embodiment bone structure 302 is a vertebral body, however, in alternate embodiments tool system 300 may be used to access other body structures such as the epiphysis, metaphysis, or diaphysis, of long bones, the subchondral bone of the tibia, femur, or humerus, and the pelvis, calcaneus, sacrum, and cranium. Vertebral body 302 includes cancellous tissue 306 and cortical bone, including pedicle 304. As depicted in the present embodiment, tool system 300 includes cannula body 310 and obturator 312. Additionally, obturator 312 includes drill section 316 and drill guide 314 as discussed above. As shown in FIG. 5A, tool system 300 has penetrated the tissue of a patient and has been advanced until drill guide 314 penetrates the exterior surface of bone structure 302. In this particular embodiment, tool system 300 is aligned to establish transpedicular access into vertebral body 302.

Now referring to FIG. 5B, following the initial placement shown in FIG. 5A, tool system 300 may preferably be rotated in the direction of arrow 320 and also urged forward in the direction of arrow 322. As tool system 300, including drill section 316 rotates, drill section 316 drills into bone structure 302, forming a bore of sufficient diameter to allow cannula 310 to advance forward into bone structure 302. The bore formed by drill section 316 is smaller in diameter than cannula 310, but facilitates controlled advancement of cannula 310 into bone structure 302. Tool system 300 may preferably be rotated and advanced forward until cannula 310 reaches a desired position. In the present embodiment tool system 300 is advanced until the distal end of cannula 310 accesses cancellous bone 306, as shown in FIG. 5C. Following the desired placement of tool system 300 within bone structure 302, obturator 312 is preferably removed, opening lumen 324 within cannula 310 and thereby providing access to cancellous tissue 306, as shown in FIG. 5D. In an alternate embodiment, cannula 310 may be a threaded cannula as described in FIG. 2, above. In such embodiments, as device 300 is rotated the threads of the cannula preferably engage bone structure 302 (specifically the walls of the bore formed by drill section 316) and control the advancement therethrough.

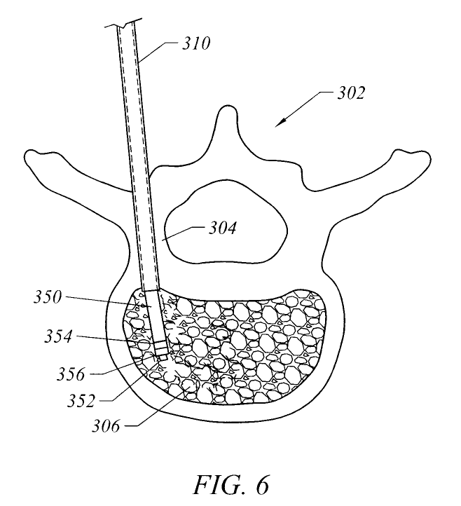

Now referring to FIG. 6, a depiction of a tool system 300 being used in a medical procedure according to teachings of the present disclosure is shown. In the present embodiment, initial cannular placement has been achieved as described above. In the present depiction, a treatment device 350 is introduced through cannula 310 to access cancellous tissue 306. In one embodiment, treatment device 350 may be a plasma-based treatment device incorporating Coblation technology as discussed above. In a particular embodiment, treatment device 350 may be a treatment device as described in U.S. patent application Ser. No. 10/970,796 which is hereby incorporated by reference. In the present embodiment treatment device 350 comprises an active electrode 352, a return electrode 354 and an insulating spacer 356. Treatment device 350 may also include a lumen for providing fluid, such as a conductive fluid as well as a suction lumen for removing said fluid and treated tissue (not expressly shown). In some embodiments, the target tissue and/or bone structure may include tumor tissue and cannula 310 may be placed to access and treat portions of said tumor tissue.

In alternate embodiments treatment device 350 may comprise any mechanical or electrosurgical device suitable for treating target tissue such as cancellous tissue 306 or tumor tissue existing within a bone structure. Treatment of target tissue may include, but is not limited to, heating, cutting, ablating and removing the target tissue.

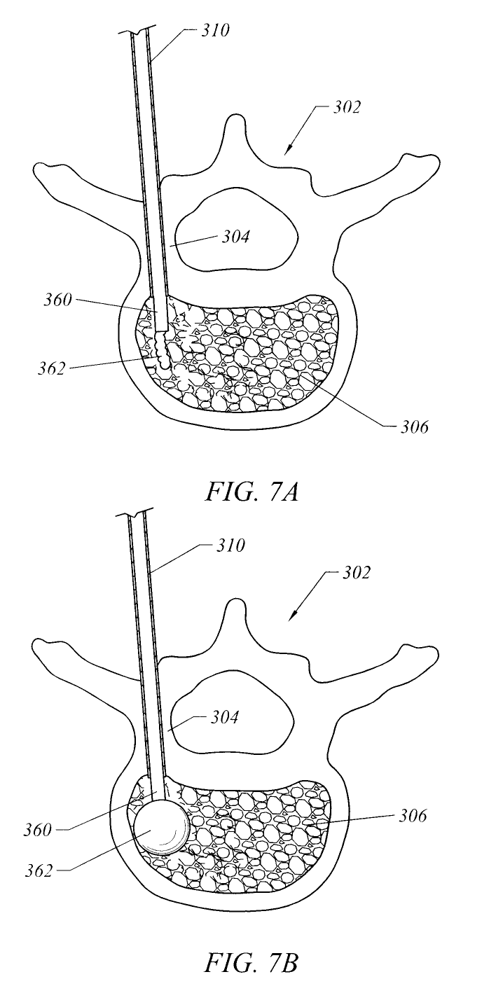

Now referring to FIG. 7A, the introduction of an expandable device 362 during a medical procedure according to teachings of the present disclosure is shown. Following placement of cannula 310 and removal of the obturator as described above, a delivery tool 360 may be inserted into cannula 310. Delivery tool 360 may be used to place expandable structure 362, within cancellous bone 306. In some embodiments, delivery tool 360 or a separate tool may be adapted to create a void in cancellous bone 306 to facilitate initial placement of the expandable structure 362 in a collapsed configuration.

Following initial placement of expandable structure 362, expandable structure is expanded, as shown in FIG. 7B. The expansion of structure 362 may be accomplished by introducing fluid into expandable structure 362 up to a selected pressure. As structure 362 expands, portions of cancellous bone 306 adjacent to the expandable structure are pushed away from expanding structure 362. Expandable structure 362 may then be collapsed and removed via cannula 310, leaving a void within cancellous bone 306.

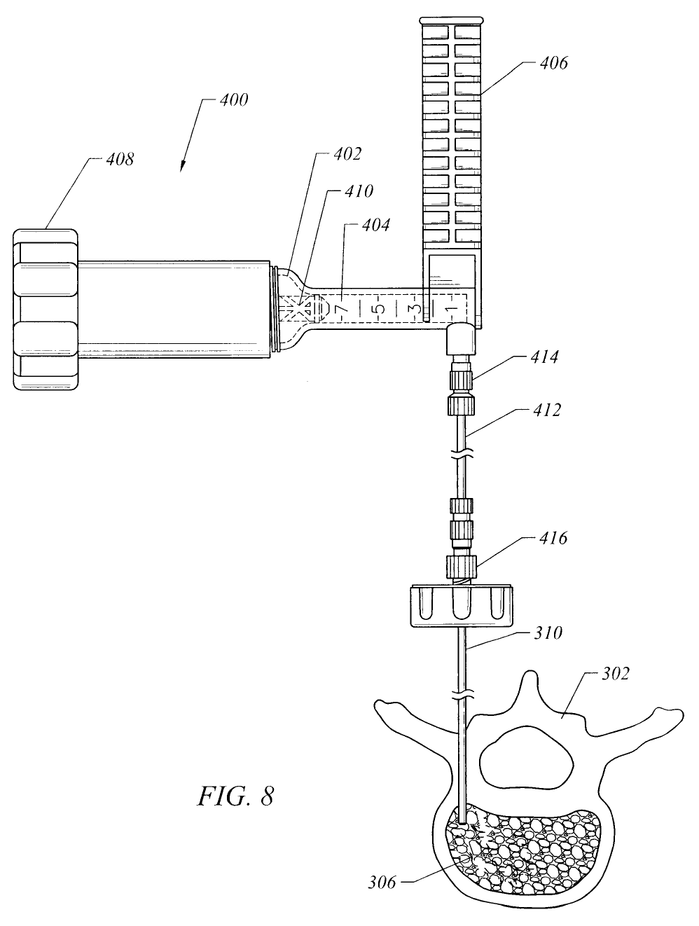

Following initial placement of cannula 310 (as shown in FIGS. 5A-5C), the treatment of cancellous tissue 306 (as shown in FIG. 6) and/or the use of an expandable device 362 (as shown in FIGS. 7A and 7B), cement or filler material may be introduced into the target site of bone structure 302. In such procedures, obturator 312 may preferably be removed from cannula 310 which is left in place at the target site. A system 400 for the controlled injection of filler material is operatively coupled to cannula 310, as shown in FIG. 8, so as to be in fluid communication with the cannula's lumen. System 400 generally includes a first column 402 and a second column 404 which holds the filler material. A handle 408 at the proximal end of first column 402 is used to drive and pressurize the filler material through column 402 and into the second column 404. Extending distally from handle 408 is a plunger head 410 for forcing the filler material through the second column 404. System 400 is in fluid communication with cannula 310 by means of a tubing 412 which is interconnected to system 400 and cannula 310 by luer locks 414 and 416, respectively. In some embodiments, tubing 412 may be a flexible conduit having sufficient length to remove a users hands from a radiographic field centered at cannula 310. A handle 406 is provided for manually handling system 400. Once system 400 is properly connected to cannula 310, the filler material is delivered to within the space created by treatment device 350 or expandable device 362 described above until a selected amount of such filler material has been injected into the space. Upon completion of the filling process, the system 400 is disconnected from cannula 310 which may then be removed from the access site, and the wound site is treated with typical care.

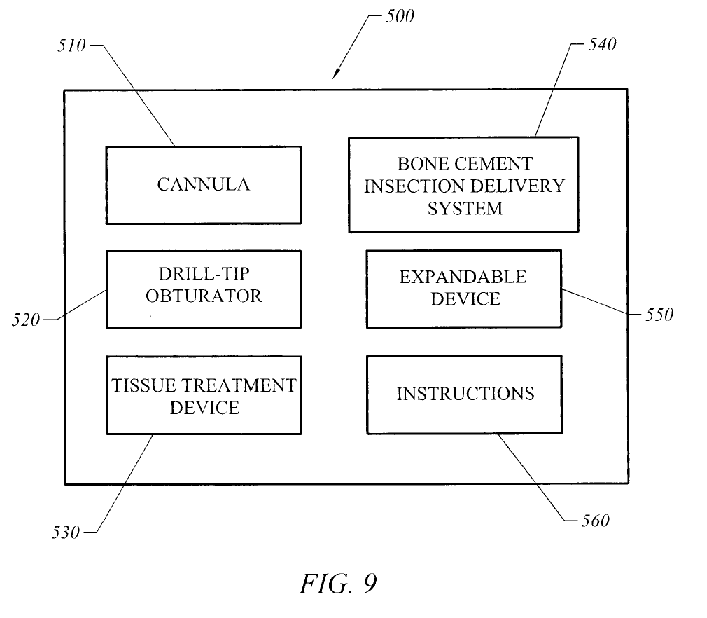

Now referring to FIG. 9, a kit, depicted generally at 500, may include component devices to perform the medical procedures as described above. FIG. 9 is a block diagram representation of a kit 500 in accordance with an embodiment of the present invention. A kit 500 may include, but is not limited to including, at least one cannula 510, at least one drill-tip obturator 520, a tissue treatment device 530, a bone cement injection delivery system 540 and an expandable device 550. It should be appreciated that multiple cannulas 510 and obturators 520 may be provided for use in a variety of applications. Also, in alternate embodiments kit 500 may be provided without, for example, a tissue treatment device 530 and/or an expandable device 550.

A subject kit such as kit 500 typically may preferably includes instructions 560 and other pertinent documentation for using the subject systems, e.g., cannula 510 and obturator 520, in methods according to the subject invention. Instructions 560 for practicing the subject methods are generally recorded on a suitable recording medium. For example, the instructions may be printed on a substrate, such as paper or plastic, etc. As such, instructions 560 may be present in the kits as a package insert or in the labeling of the container of the kit or components thereof, i.e., associated with the packaging or subpackaging. In other embodiments, instructions 560 may include electronic data stored on a suitable computer readable storage medium, e.g., CD-ROM, DVD, diskette, etc. In yet other embodiments, the actual instructions are not present in the kit, but means for obtaining the instructions from a remote source, e.g., via the Internet, are provided in lieu of instructions 560. An example of this embodiment is a kit that includes a web address where the instructions can be viewed and/or from which the instructions can be downloaded. As with the instructions, this means for obtaining the instructions is recorded on a suitable substrate.

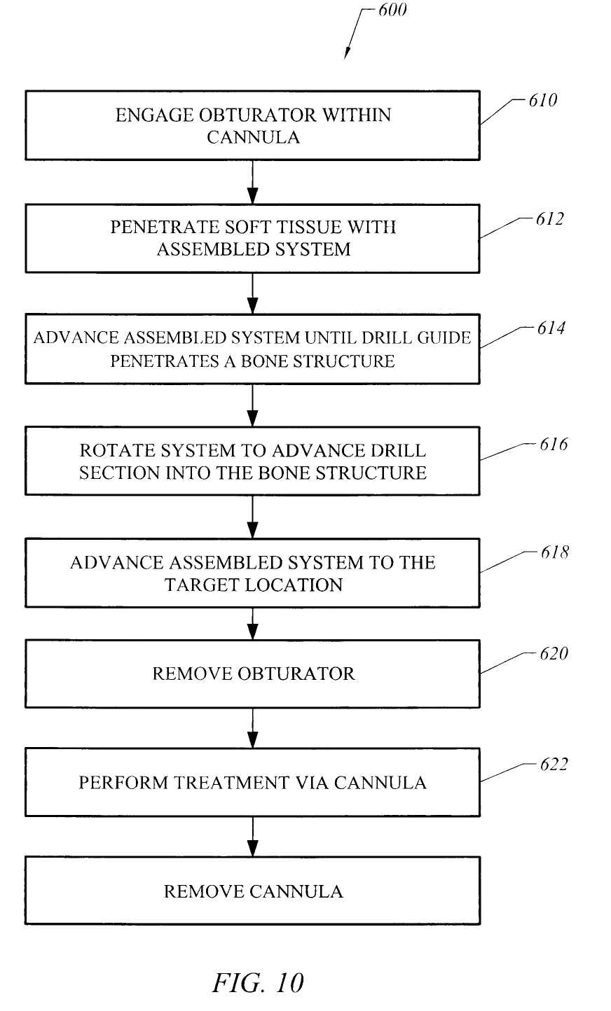

With reference to FIG. 10, one method of performing a medical procedure using a cannula and obturator according to an embodiment of the present invention is shown. It should be appreciated that various steps which are typically included in a vertebroplasty procedure or kyphoplasty procedure are not described for ease of discussion. Such steps may include, but are not limited to, the anesthetization of skin in the vicinity of the bone structure which is to be penetrated, the preparation of bone cement, the verification of the proper positioning of the tip of the cannula, and the determination of when sufficient bone cement has been injected into an implantation site.

A process 600 of performing a medical procedure begins at step 610 wherein obturator (such as obturator 30 as shown in FIG. 1) is inserted with a cannula (such as cannulas 12 and 112. Next, the assembled cannula and obturator are used to penetrate the soft tissue of a patient 612. The assembled system is then advanced until the drill guide penetrates a selected bone structure 614. The assembled system may then preferably be rotated to advance the drill section of the obturator into the bone structure 616 and advanced into the bone structure until a desired placement has been achieved 618 to access a selected target tissue. If a threaded cannula is used (as shown in FIG. 2) the threaded portion of the cannula and the rotation of the system may facilitate the translation of the system into the target bone structure and selective placement of the system. Following placement of the system, the obturator may be removed 620 and the target tissue may be treated through the cannula 622. As discussed above, treatment of the target tissue may include: removal of tissue, forming a void within the target tissue using an expandable device, injecting filler material (such as bone cement) into the target bone structure, or any combination thereof. Following treatment of the target tissue, the cannula is removed from the patient 624. In a preferred embodiment the above steps may be visually assisted by fluoroscopy or other imaging techniques known in the surgical arts.

Although only a few embodiments of the present invention have been described, it should be understood that the present invention may be embodied in many other specific forms without departing from the spirit or the scope of the present invention. Therefore, the present examples are to be considered as illustrative and not restrictive, and the invention is not to be limited to the details given herein, but may be modified within the scope of the appended claims.

What is claimed is:

1. An obturator comprising:
an elongated body having a longitudinal axis, a diameter, a proximal end and a distal end;
a drill section forming a portion on the distal end of the elongated body; and
a drill guide extending from the distal end of the elongated body and adapted to penetrate a bone structure.

2. The obturator of claim 1 wherein the drill guide has a substantially conical configuration.

3. The obturator of claim 1 wherein the drill guide comprises a pointed distal end and a proximal end having a first a diameter, wherein the first diameter is smaller than the elongated body diameter.

4. The obturator of claim 1 wherein the drill guide comprises a tip having a diamond configuration.

5. The obturator of claim 1 wherein the drill guide extends substantially along the longitudinal axis.

6. The obturator of claim 1 wherein the drill portion further comprises at least one flute formed in the outer surface of the elongated body.

7. The obturator of claim 6 wherein the at least one flute comprises a helix angle of about 20°.

8. The obturator of claim 6 wherein the at least one flute comprises a helix angle between about 15° and about 25°.

9. The obturator of claim 1 wherein the drill portion comprises a length between about 0.25 inches and about 0.75 inches.

10. The obturator of claim 1 further comprising an obturator handle disposed at the proximal end of the elongated body.

11. The obturator of claim 1 further comprising a drill point extending from a distal end of the drill portion to a proximal end of the drill guide.

12. The obturator of claim 11 further comprising the drill point having a drill point angle between about 90° and 150°.

13. The obturator of claim 11 further comprising the drill point having a drill point angle between about 115° and 125°.

14. The obturator of claim 11 further comprising the drill portion adapted for drilling into a bone structure.

15. A tool system comprising:
a cannula; and
an obturator having an elongated body, the elongated body having a proximal end and a distal end, a drill section formed on a portion of the distal end of the elongated body and a drill guide extending from the distal end of the elongated body, the obturator adapted to fit within and be removed from the cannula.

16. The tool system of claim 15 wherein the cannula comprises a proximal end and a distal end, the distal end having at least one thread formed thereon and adapted for being driven into a bone structure.

17. The tool system of claim 15 wherein:
the cannula comprises a cannula handle attached to a proximal end of the cannula;
the obturator comprises an obturator handle attached to the proximal end of the obturator; and
the obturator handle adapted to be removably coupled with the cannula handle.

18. The tool system of claim 15 wherein:
the cannula comprises a proximal end, a distal end and a first length; and
the elongated body comprises a second length selected to allow the elongated body to fit within the cannula and allow at least a portion of the drill section to extend from the distal end of the cannula.

19. The tool system of claim 15 wherein the drill guide comprises a pointed distal end.

20. The tool system of claim 15 wherein the drill section further comprises at least one flute.

21. The tool system of claim 15 wherein the obturator further comprises a drill point extending from the distal end of the drill portion and terminating at a proximal end of the drill guide.

22. The tool system of claim 15 further comprising the obturator drill portion adapted for drilling into a bone structure.

23. A medical procedure to be performed on a body comprising:
slidably engaging an obturator within a cannula, wherein the obturator comprises an elongated body, the elongated body having a proximal end and a distal end, a drill section formed on a portion of the distal end of the elongated body and a drill guide extending from the distal end of the elongated body;
penetrating tissue with the assembled cannula system;
advancing the assembled cannula system until the drill guide penetrates a bone structure; and
advancing the assembled cannula to a target location within the bone structure by rotating the cannula system, thereby causing the drill portion to drill into the bone structure.

24. The medical procedure of claim 23 further comprising removing the obturator from the cannula.

25. The medical procedure of claim 24 further comprising:
inserting a distal end of a tissue removal device in the cannula; and
removing tissue from the target location.

26. The medical procedure of claim 24 further comprising:
inserting an expandable structure in the cannula;
locating the expandable structure within the target location; and
causing the expandable structure to assume an expanded geometry.

27. The medical procedure of claim 24 further comprising injecting a bone cement into the bone structure.

28. The medical procedure of claim 23 further comprising applying a rotational motion to the cannula to adjust the depth of insertion of the cannula with respect to the bone structure, wherein the cannula comprises a threaded cannula.

29. The medical procedure of claim 23 wherein the bone structure is a vertebral body.

30. A system for accessing a body structure comprising:
a cannula for insertion within a body structure;
an obturator adapted for slidable engagement and removal from the cannula, the obturator comprising:
a drill guide means extending from the obturator adapted for engaging a body structure; and
a drill means for boring into the body structure, the drill means disposed proximate the drill guide means.

31. A kit comprising:
a cannula;
an obturator adapted to fit within and be removed from said cannula, the obturator comprising an elongated body having a proximal end and a distal end, a drill section formed on the distal end of the elongated body and a drill guide extending from the distal end of the elongated body; and
a bone cement injection delivery system having a connector adapted to fluidly connect said delivery system to said cannula such that bone cement may be delivered from said system through said cannula to a target site.

32. The kit of claim 31 further comprising an expandable device adapted to be delivered to the target site through the cannula and expanded within the target site.

33. The kit of claim 31 further comprising a tissue removal device having a distal ended adapted to fit within the cannula and perform tissue removal at the target site.
